(19) United States
(12) Patent Application Publication
(10) Pub. No.: US 2003/0109806 A1
(43) Pub. Date: Jun. 12, 2003

(54) Reservoir for flood withdrawal

(76) Inventors: Jorg Weber, Edling (DE); Peter Pfandl, Haar (DE)

Correspondence Address: DOUGHERTY, CLEMENTS & HOFER, 1901 ROXBOROUGH ROAD, SUITE300, CHARLOTTE, NC, 28211, US

(21) Appl. No.: 10/315,351

(22) Filed: Dec. 10, 2002

(30) Foreign Application Priority Data
Dec. 11, 2001 (DE) 101 60 703.2

(51) Int. Cl. A61B 5/00; B65D 81/00

(52) U.S. Cl. 600/578

(57) ABSTRACT

The reservoir for blood withdrawal has a housing (1) and a displaceable piston (7) located therein by means of which, in the interior of the housing, a reservoir chamber (16) of variable size can be formed. A piston is coupled to an actuation device (11). Furthermore, the piston (7) is pressed by a first spring (14) into a closed position in which the reservoir chamber (16) has a minimum volume. The actuation device (11) and the piston (7) are loosely coupled to each other by a second spring (10), where the spring excursion of this spring (10) is smaller than the movement path (13) of the actuation device, so that, at the end of the movement, a forced coupling between the piston and the actuation device (11) occurs.

### DESCRIPTION

The invention relates to a reservoir for blood withdrawal according to the preamble of claim 1. such a reservoir is known for example from DE 297 13 743 U1.

Patients into whom an infusion tube or a catheter has been inserted, in a vein or artery, must frequently have a blood sample taken for examination purposes, and this procedure is advantageously carried from a site of the infusion tube which is accessible from the outside. For this purpose, a blood withdrawal site is provided in the infusion system. Before blood withdrawal can take place, one must ensure that, in the area of the blood withdrawal site only undiluted blood of the patient is present, and no portions of infusion solutions or diluents.

WO 88/01846 therefore provides for two blood withdrawal sites The blood withdrawal site located closer to the patient, and in an anterior position, is used for drawing the blood sample proper, and the posterior withdrawal site located at a distance from the patient serves the function of temporarily removing infusion solutions from the system so that only undiluted blood is present in the anterior withdrawal site. This second withdrawal site thus has the function of a reservoir. After blood withdrawal at the anterior withdrawal site, the quantity which has temporarily been stored in the reservoir is then again infused into the patient.

DE 297 13 743 U1 shows a reservoir with a chamber connected to a branch of an infusion tube. The system can be moved in this chamber and it can suck blood and/or infusion solution into the chamber as it is pulled back. The piston rod of the piston is attached to a handle, where a spring which presses the handle and thus the piston in a direction for blood withdrawal is provided between the handle and a support in the chamber, so that the suction of blood or infusion solution into the chamber occurs substantially automatically. The user only needs to undo the latch at the handle. Similar blood withdrawal devices with a spring which effects an automatic aspiration can also be obtained from DE 94 19 737 U1 and DE 40 19 197 C2. In general, for blood withdrawal sites of this type, i.e., reservoirs, reference is also made to DE 42 20 301 C1 and EP 0 301 913 A2.

An advantage of the automatic blood withdrawal devices based on spring force consists in that it can be operated with one hand. However, this also entails a risk, because as a result of the low pressure produced during the aspiration, small gas bubbles can form which later reach the circulation of the patient during reinfusion. This is particularly the case if the actuation handle is accidentally unlatched and aspiration occurs when certain valves in the infusion lines are closed.

The problem of the invention is to improve the reservoir of the type mentioned above so that it offers higher reliability and simpler operation.

This problem is solved by the characteristics indicated in claim 1. Advantageous configurations and developments of the invention can be obtained from the secondary claims. A spring is also used in the invention, however, it is used for emptying the reservoir, not for aspiration. This increases reliability because undesired aspiration is no longer possible, while the emptying of the reservoir is simplified. To further increase reliability, the piston is uncoupled from the actuation device by an additional spring. By means of the temper of this additional spring, it is then possible to compensate for the maximum possible low pressure which can be generated when the aspiration takes place and the valves are closed.

In a development of the invention, the reservoir, which is “inline” with respect to the infusion tube, also has a separated inlet and outlet connected by a pipe to each other. Advantageously, this pipe is rigid and it is arranged essentially in the housing, so that the displaceable piston runs along this pipe. However, it is mentioned that the blood withdrawal device can also be connected in a known manner laterally to an infusion line, for example according to DE 42 20 301 C1.

The invention is explained in greater detail below with reference to an embodiment example in connection with the drawing.

The single FIGURE shows a cross section of the reservoir.

This reservoir has an essentially cylindrical housing 1 which has connections 2 and 3 at both axial ends to which the tube sections 4 and 5 can be connected. In the interior of the housing, a rigid pipe 6 is centrally arranged, which opens immediately into the tube 4 at connection 2 away from the patient, while, at the end on the patient side, close to connection 3, it opens in chamber 16. In the interior of the housing, a displaceable piston 7 is arranged which leads to the rigid pipe 6 and which is applied to the interior wall of the housing 1 in a manner so it provides a seal.

The piston 7 is connected with first catch 8, which here has an approximately rectangular cross section and a lower-opening 8.1 for the passage of the pipe 6, as well as an upper opening 8.2 for the passage of a second catch 9. The lower wall 8.3 of the first catch 8 is rigidly connected to the top side of the piston 7, for example by gluing.

A second catch 9, whose cross section is in the approximate shape of a double T, extends with an axial leg 9.1 through the opening 8.2 of the first catch in its interior, where it ends at a first transverse leg 9.2, on which the second spring 10 is supported. The other end of the second spring 10 is supported by the upper interior wall 8.4 of the first catch. The second transverse leg 9.3 of the second catch is located outside of the first catch. On it, a first spring 14 rests, whose other end is applied to a spring support 17 which is arranged at the end of the housing 1 close to the connection 2. The second catch 9 presents a pin which protrudes to the outside through a slit 13 into the housing 1, where it is connected with an actuation device such as, e.g., a ring 11 or a head. At the top end of slit 13, i.e. the end close to connection 2, a bayonet locking mechanism 13.1 is provided in which the pin can engage.

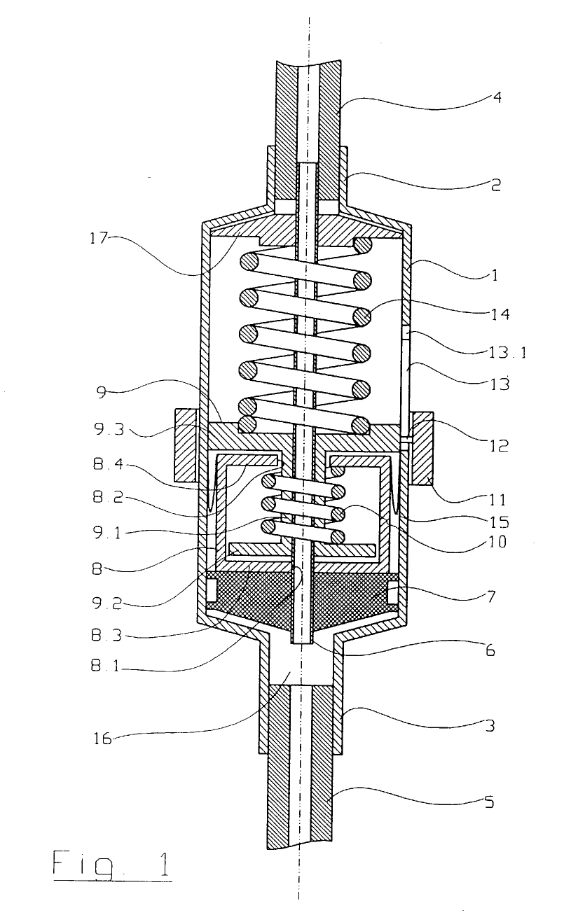

The operating procedure of the device is described below; starting at the “rest position” represented in FIG. 1, the actuation device 11 is pulled manually upward, i.e. in the direction toward connection 2. In the process, the second catch 9 is shifted against the force of the spring 14, and the transverse leg 9.2 of the second catch drives the first catch 8 with the second spring 10, which catch in turn then displaces the piston 7, so that the volume of the chamber 16 is increased. As a result, fluid is sucked from the hose 5 into the chamber 16, where it is temporarily stored. The pin 12 reaches the upper end of the slit 13, then it can be engaged, by movement in the circumferential direction, in the bayonet locking mechanism 13.1, and the device stores the corresponding volume in chamber 16 which is opened to a maximum.

As is conventional with such blood withdrawal devices, prior to the beginning of the suction a valve, not shown, is closed in tube 4 so that fluid is sucked out of only one tube 5.

The actuation device 11 and the second catch 9 are decoupled with a respect to the piston 7 by means of the second spring 10. The piston thus does not follow the movement of the second catch immediately, but as a function of the chosen temper of the second spring 10, and only to such an extent that the low pressure in chamber 16 does not exceed a predetermined level.

The reinfusion of the fluid and thus the emptying of the chamber occur in the reverse order. If the pin 12 is separated from the bayonet locking mechanism 13.1, then the first spring 14 presses the second catch down and the latter in turn presses the first catch 8 directly via the bar 8.4, thus pressing piston 7 down. In this direction the two catches are directly coupled, because no decoupling is required for the reinfusion.

Finally it is noted that between one of the two catches 8 and 9 and the interior wall of the housing 1, a seal in the form of a roll tube 15 is provided, which is arranged so it is adjacent to the slit 13 in the direction toward the connection 3 on the patient side, and thus it prevents contamination from reaching the part of the housing 1 through slit 13, which part is connected with chamber 16. Naturally, the roll tube surrounds the first catch 8.

Furthermore, it is also mentioned that the connection 2 advantageously is formed as a rigid connection with respect to tube 4, whereas connection 3, with respect to the hose, functions as a connection which can be loosened, for example a Luer coupling or a screw coupling.

The separation between the transverse leg 8.4 and 9.2, as well as the thickness of the spring 10 in the completely compressed state are smaller than the length of the slit 13, and thus one ensures that the piston 7 can always be loosened from its forward position, even if it is attached there by gluing, for example due to blood residues. In the last phase of the movement of the catch 9.3, a forced coupling is thus provided, because the spring excursion of the spring 10 has been completely covered, and the spring then no longer has a spring action, rather it acts as a rigid part.

1. Reservoir for blood withdrawal having a housing (1) defining a chamber (16), a piston (7) in said housing movable by an actuation device (11), one direction of movement of the piston (7) being supported by a spring (14),the spring (14) being adapted to press the piston (7) into a closed position in which the chamber (16) of the housing (1) has its minimum volume.

2. Reservoir according to claim 1, characterized in that the piston (7) and the actuation device (11) are coupled to each other by a second spring (10).

3. Reservoir according to claim 2, characterized in that the piston (7) is rigidly connected to a first catch (8), the actuation device (11) is rigidly connected to a second catch (9), and
the first and second catches (8 and 9) are coupled to each other by means of the second spring (10) in such a manner that, during an opening movement of the piston (7), the second catch (9) moves, by means of the second spring (10), the first catch and thus the piston.

4. Reservoir according to claim 3, characterized in that the spring excursion of the second spring is smaller than the maximum possible movement path of the second catch 9, so that at the end of the spring stroke excursion of the second spring (10), a forced coupling between the actuation device (11) and the piston (7) occurs.

5. Reservoir according to claim 1, characterized in that the housing (1), in the axial direction, presents a first connection (2) and a second connection (3), and a rigid pipe (6) is centrally located with respect to the axis of the housing (1), where one end of the pipe is connected to the first connection (2) and the other end opens into the chamber (16).

6. Reservoir according to claim 1, characterized in that, the housing is provided with a longitudinal slit (13) the actuation device (11) penetrates through which via a pin (12) to which the second catch (9) is connected.

7. Reservoir according to claim 6, characterized in that the slit (13) presents a bayonet locking mechanism (13.1) for the pin (12).

8. Reservoir according to claim 1, characterized in that, for the action, a roll seal (15) is provided between the internal wall of the housing (1) and the piston (7).

9. Reservoir according to claim 8, characterized in that the roll seal (15) is attached, on the one hand, to the internal wall of the housing (1) and, on the other hand, to the first catch (8) or to the second catch (9).
